(19) United States
(12) Patent Application Publication
(10) Pub. No.: US 2021/0025798 A1
(43) Pub. Date: Jan. 28, 2021

(54) APPARATUS AND METHOD FOR MEASURING THE TRANSPIRATION OF PLANTS

(71) Applicants: Jin-Chen Liu, Dachau (DE); Michael Martin Goisser, Freising (DE)

(72) Inventors: Jin-Chen Liu, Dachau (DE); Michael Martin Goisser, Freising (DE)

(21) Appl. No.: 16/933,228

(22) Filed: Jul. 20, 2020

(30) Foreign Application Priority Data
Jul. 22, 2019 (DE) DE 102019210818.0

(51) Int. Cl. G01N 5/04; G01K 7/02; G01K 3/14; G01K 1/02; G01N 33/00

(52) U.S. Cl. CPC G01N 5/045; G01K 7/02; G01N 33/0098; G01K 1/026; G01K 3/14

(57) ABSTRACT

The present disclosure relates to an apparatus and a method for ascertaining a transpiration rate of an object, in particular of a plant leaf or a plant needle. The method comprises the steps of ascertaining a first temperature difference between the temperatures on the surface of the object and on the surface of a reference body as well as a second temperature difference between the temperatures on the surface of the object or on the surface of the reference body and a respective measurement point (M1, M2) spaced apart therefrom, so as to finally calculate therefrom the transpiration rate of the object.

### CROSS-REFERENCE TO RELATED APPLICATION

This application claims priority to German Patent Application No. DE102019210818.0, filed Jul. 22, 2019, the contents of which are hereby incorporated by reference in their entirety.

### BACKGROUND

The present disclosure relates to an apparatus for ascertaining a transpiration rate of an object, e.g. of a plant leaf or a plant needle, and to a corresponding method.

Transpiration is an essential physiological process in plants. The transpiration rate (i.e. the amount of water transpired over a certain period of time) is a basic parameter in the water balance of plants and is therefore of great interest for applications such as irrigation control, smart farming and research. Transpiration rates are strongly dependent on the plant species and the irrigation status of the plant. Typical transpiration rates range from 0.2-4.0 mmol*m\hat{ }-2*s\hat{ }-1 for beech and 0.1-1.0 mmol*m\hat{ }-2*s\hat{ }-1 for spruce.

Transpiration can be measured with different methods. The simplest one is, for example, the lysimeter. Here, the change of weight of the plant, which stands together with the plant pot on a scale, is determined continuously. The loss of weight is regarded as transpiration.

Another important method is the determination of xylem flow (U.S. Pat. No. 4,745,805, DE 102 22 640, U.S. Pat. No. 5,269,183 A).

Both lysimeter and xylem flow indicate the transpiration of the plant in its entirety. However, knowing the direct plant-derived regulation of water consumption at the actual place in question, i.e. on the leaf level, is of great value to the above-mentioned applications. For methodological reasons, more precise information on transpiration on the leaf level can, however, be derived neither from lysimeter data nor from xylem flow data.

The most common method for determining water release on the leaf level is the gas exchange measurement making use of a gas cuvette. The respective transpiration organ or object (leaf, needle or bark) is here enclosed in a transparent vessel. The increase in humidity in the air or in the exhaust air of the vessel is determined, and the transpiration per leaf area can be calculated from the value determined (see DE3032833C2). The disadvantage of this method is the significant change in the natural physical environment of the transpiration organs under consideration. Due to this change in the ambient physical conditions, a large deviation from the actual transpiration cannot be avoided.

A known method, which, however, has been given little consideration in the past, is the measurement of the temperature difference between the leaf and a non-transpiring reference body (Delta-T(ref-leaf)). During transpiration, water in the leaf turns into gaseous form and thereby cools the leaf, whereas such cooling down does not take place in the reference body. Hence, the difference in temperature between the two reflects the intensity of transpiration (Impens. I. et al.: Diffusive resistances at, and transpiration rates from leaves in situ within the vegetative canopy of a corncrop. Plant physiology, 1967, 42 volume, No. 1, pp. 99-104).

For measuring the temperature difference, e.g. thermopiles are used (Haines, F. M.: Transpiration and Pressure Deficit, Ill. Observations by the Thermopile Method. Annals of Botany, 1936, No. 1, pp. 1-22). In recent years, temperature measurement has been carried out by means of thermography (Garbe, C., Schurr, U., & Jaehne, B.: Thermographic measurements on plant leaves. 2002, Proc. SPIE, Vol. 4710, pp. 407-416).

The Delta-T(ref-eaf) method causes only little disturbance in the ambient natural conditions and thus allows the continuous measurement of transpiration under realistic conditions. Another important advantage of the method is its comparatively simple and cost-effective implementation. Despite the above-mentioned advantages, this method is, however, not widely used in practice. One reason for this is the complex interpretation of the measurement signal due to the interaction of the measurement signal with environmental factors, in particular wind on the leaf surface. It is obvious that Delta-T(ref-leaf) is influenced by wind. At the same transpiration rate, stronger wind will lead to a smaller temperature difference Delta-T(ref-leaf). The practical application of the Delta-T(ref-eaf) method is therefore only possible in cases where the wind speed on the leaf surface is additionally measured. So far, however, this has not been possible with reasonable effort.

The aim of the present disclosure is to be seen in providing, with the simplest possible means in terms of construction, an apparatus and a method for reliably ascertaining a transpiration rate of an object.

This aim is achieved by an apparatus having the features of claim 1 or by a method having the features of claim 5. Advantageous further developments of the present disclosure are specified in the dependent claims.

The present disclosure is based on the surprising idea of ascertaining, in addition to a first temperature difference for comparing the object (or sample) and the ideally transpiration-free reference body, a second temperature difference between the temperatures on the surface of the object or of the reference body and a measurement point spaced apart therefrom to a certain extent. This second temperature difference is now used as a measure proportional to the wind velocity on the surface of the object, so as to be able to determine the transpiration rate of the object on the basis of the two measured temperature differences. The data recorder used for recording or calculating the first and second temperature differences may be a computer. Alternatively, the data recorder could exclusively provide a recording function and could e.g. be connected to a computer for evaluating the data.

Preferably, the apparatus comprises temperature sensors, in particular thermistors, for measuring the temperatures.

Alternatively, a respective thermocouple, such as a thermopile, may, however, be used for measuring the first and/or second temperature difference, this kind of thermopile being described e.g. in DE 10 2015 120 899 A1. Hence, the present disclosure provides the possibility of directly measuring temperature differences, instead of measuring absolute temperatures.

According to an advantageous embodiment, a compensating body is thermally connected to the thermopile. This compensating body should have a medium to high thermal conductivity. It allows to measure, instead of a local temperature, an average temperature of a larger area of the surface of an object, and this may be of advantage, in particular for objects with inhomogeneous thermal properties.

The present disclosure also relates to a method for ascertaining a transpiration rate of an object. The method comprises the following steps:
determining a first temperature difference between the temperatures on a surface of the object and on a surface of a reference body,
determining a second temperature difference between the temperatures on the surface of the object and at a first measurement point spaced apart from the surface of the object, or between the temperatures on the surface of the reference body and at a second measurement point spaced apart from the surface of the reference body,
ascertaining the transpiration rate of the object, taking into account the first and the second temperature difference.

Hence, the second temperature difference is, so to speak, used as an indicator for the wind velocity at the boundary between the surface of the object or of the reference body and the ambient air, so that the transpiration rate of the leaf can then be ascertained in combination with the first temperature difference independently of the respective wind velocity prevailing.

It will be advantageous when the transpiration rate of the object is ascertained taking into account a calibration. Such calibration may, for example, be carried out on the basis of a gravimetric measurement of the transpiration or of the transpiration rate of the object or of a comparison object. For this purpose, it would be imaginable to place the measurement setup on a scale that is as precise as possible, in order to measure the water loss and thus the transpiration and simultaneously the two temperature differences gravimetrically (i.e. through the change of weight). From the change or the development with time of these measured data, the parameters of the transpiration can then be determined deterministically or statistically, by way of example.

During calibration, the gravimetrically ascertained transpiration loss of the leaf/needle branch (in kg water per unit time, converted into substance quantity mol, divided by molar mass H₂O) may, for example, be compared with the continuously recorded sensor data.

It proved to be advantageous when the first measurement point is spaced apart from the surface of the object by a maximum distance of 10 cm, or when the second measurement point is spaced apart from the surface of the reference body by a maximum distance of 10 cm. In this way it is ensured that a measure of the wind velocity at the location of the object and of the reference body, respectively, will actually be taken into account.

The method will be particularly suitable, when the object used is a plant leaf or a plant needle. However, the method may also be applied to other transpiring objects.

For particularly precise measurements, it will be of advantage when the object and the reference body are as similar as possible with respect to their geometry, their light characteristics and/or their heat characteristics. In concrete terms, this can be accomplished, when the object and the reference body differ from each other with respect to a dimension, with respect to a volume, with respect to an absorption coefficient at a specific wavelength or in a specific wavelength range, with respect to their thermal capacity and/or with respect to their coefficient of thermal conductivity by a maximum of 20% in each case, preferably by a maximum of 10%.

### BRIEF DESCRIPTION OF THE DRAWINGS

In the following, advantageous embodiments of the present disclosure will be explained in more detail on the basis of a drawing. The individual figures show

FIG. 1 a schematic representation of a first embodiment;

FIG. 2 a schematic representation of a second embodiment;

FIG. 3 a schematic representation of a third embodiment; and

FIG. 4 a schematic representation of a fourth embodiment;

### DETAILED DESCRIPTION

Like components are provided with like reference numerals throughout the figures.

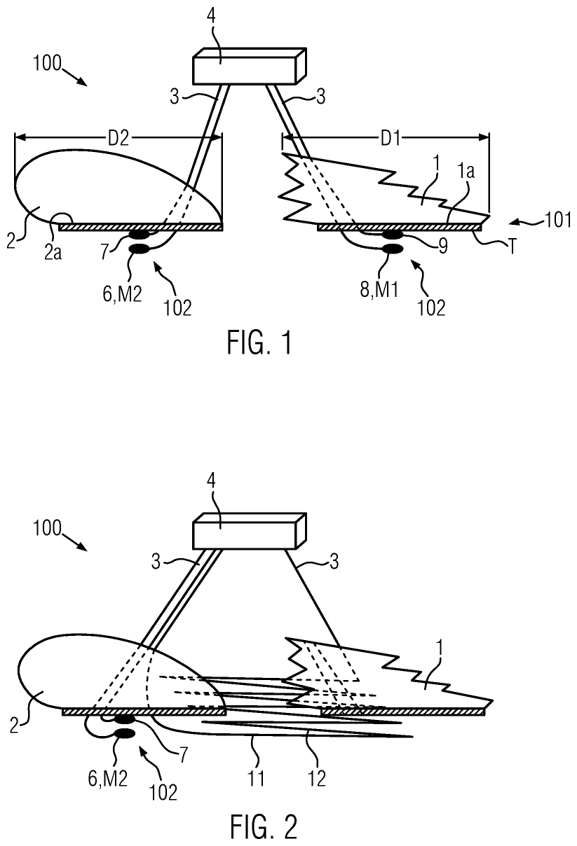

FIG. 1 shows a first apparatus 100 for ascertaining a transpiration rate of an object 1, which is a plant leaf 1 that may e.g. lie on or be attached to a carrier T.

A reference body 2, which is similar to the leaf 1 as regards geometry as well as light and heat characteristics, but which does not transpire, is placed at a distance of a few millimeters to a maximum of approx. 50 cm from the leaf 1. In the context of the present disclosure, the phrase “similar to the leaf as regards geometry as well as light and heat characteristics” means that the object 1 and the reference body 2 differ from each other with respect to a dimension, with respect to an absorption coefficient at a specific wavelength or in a specific wavelength range, with respect to a thermal capacity of their material and/or with respect to a coefficient of thermal conductivity of their material or their surface by a maximum of 20% in each case, preferably by a maximum of 10%. For example, a dimension D2 of the reference body 2 may deviate by up to 20% from a corresponding dimension D1 of the object 1. The deviation may, for example, be considered taking as a basis the respective object 1, 2 in the case of which the size in question has the larger value. The aim is that the reference body 2 and the object 1 are as similar as possible with regard to one or more characteristics. This, however, does not apply to transpiration. Ideally, the reference body 2 is not transpiring at all, i.e. it does not contain any water, by way of example. In the embodiment according to FIG. 1, the reference body 2 is placed on a carrier of its own, which, however, is optional or which may also be the same carrier T for the object 1.

The temperatures at the leaf 1 (Tl), at the reference (Tr), as well as the temperature close to the leaf (Tla) or close to the reference (Tra) are now measured preferably in parallel. The Delta-T(ref-leaf) results from the difference between the temperatures Tl and Tr. The temperature difference between Tl and Tla as well as Tr and Tra shows the intensity with which the air at the boundary layer is removed by the wind. Hence, it is an indicator of the wind velocity at the boundary surface/ambient air of the leaf 1 and of the reference 2, respectively. The transpiration rate of the leaf 1 can be calculated with the following equation:

W=f(Delta-T(ref-leaf),(Tr−Tra)) or

W=f(Delta-T(ref-leaf),(Tl−Tla))

The parameters of these equations can be ascertained gravimetrically prior to the actual measurement, e.g. in the form of a calibration. When the setup is placed on a very precise scale and the transpiration and the four temperatures are thus measured gravimetrically (i.e. via the weight loss over a certain period of time) in parallel, the parameters can be determined deterministically or statistically, by way of example.

A concrete example of such an equation could be as follows:

W=a+b*(Tr−Tl)+c*(Tl−Tla)+d*(Tr−Tra)

Parameters for the calibration function, ascertained via gravimetric comparative measurement on spruce, under variable ambient conditions (radiation, air temperature, humidity)

Coefficient of determination of the calibration function Rsq: 94.4%

a=+0.000274455
b=+0.452055039
c=−0.005186585
d=−0.000632594

The apparatus 100 in FIG. 1 comprises a first arrangement 101 for determining a first temperature difference between the temperatures on the surface 1a of the object 1 and on the surface 2a of the reference body 2. This first arrangement 101 comprises a first temperature sensor 9, which contacts the carrier T or even better directly the surface 1a of the object 1 and measures the temperature Tl of the latter, and a second temperature sensor 7, which directly measures the temperature on the surface 2a of the reference body 2. Furthermore, the apparatus 100 comprises a second arrangement 102 for determining a second temperature difference. For this purpose, two different variants are shown in FIG. 1. In one variant, the second arrangement 102 comprises the first temperature sensor 9 for measuring the temperature on the surface 1a of the object 1 and a further temperature sensor 8 at a first measurement point M1. The first measurement point M1 is here spaced apart from the first temperature sensor 9, the distance being, however, preferably not larger than 10 cm. In the case of this arrangement 102, the second temperature difference ascertained is the difference between the temperatures measured by the two temperature sensors 8, 9.

In a second variant, the arrangement 102 comprises the temperature sensor 7 for measuring the temperature on the surface 2a of the reference body 2 as well as a further temperature sensor 6, which measures the temperature at a second measuring point M2. This second measuring point M2 is spaced apart from the temperature sensor 7 and the surface 2a of the reference body 2, the distance being, however, preferably not larger than 10 cm.

The temperature difference between leaf 1 and reference 2 Delta-T(ref-leaf) is measured in FIG. 1 with temperature sensors 7 (Tr) and 9 (Tl). The temperature close to the leaf (Tla) and the temperature close to the reference (Tra) are measured with two further temperature sensors 6, 8. The data are transmitted to the data recorder 4 via a cable 3. The temperature recorder 4 may be a computer.

FIG. 2 shows a further possible embodiment. Instead of using temperature sensors for an absolute measurement, a thermopile 11 with windings 12 is here used for a direct measurement of the temperature difference. The thermopile 11 records several measurement points and thus takes into account the thermal inhomogeneity of the leaf 1. The temperature difference between the reference body 2 and the air close to the reference (Tr−Tra) is measured with two thermistors used as temperature sensors 6 and 7.

It is obvious that the temperature sensors 7 and 6 may also be placed on the side of the leaf 1. Due to the different thermal characteristics, the calibration curve will then be different.

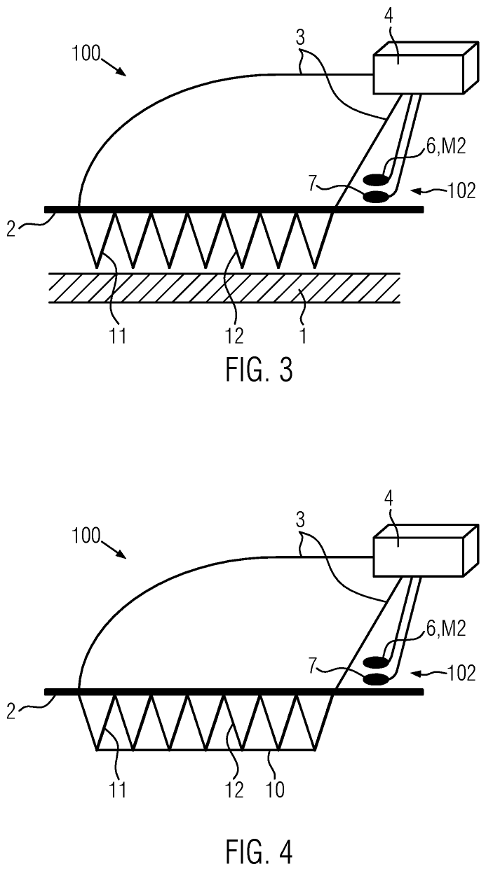

Analogously to the sensor for a leaf, a similar sensor may be produced for plant species with needle leaves 1 as an object. FIG. 3 shows one of the possible variants for needle leaves. Here, the measurement points of the thermopile 11 with windings 12 are arranged on the reference body 2, which has thermal characteristics similar to those of the needles 1. The other side of the thermopile 11 is inserted into the needle tuft 1, it measures the temperature in the interior of the needle tuft 1 at several points. The absolute temperature at the reference (Tr) and in the air close to the reference (Tra) is measured by temperature sensors 6 and 7.

For leaves 1 and needles 1 having an inhomogeneous thermal characteristic, the thermopiles 11 in FIGS. 2 and 3 can only with difficulty precisely measure the correct leaf temperature. In this case, a compensating body 10 is attached to one side of the leaf, as can be seen in FIG. 4. The compensating body 10 has the best possible thermal conductivity and reflects the average temperature of the leaf 1 or the needle 1, and transmits this to the thermopile 11.

1. An apparatus for ascertaining a transpiration rate of an object, in particular of a plant leaf or a plant needle, the apparatus comprising:
a reference body, an arrangement for determining a first temperature difference between the temperatures on a surface of the object and a surface of the reference body, an arrangement for determining a second temperature difference between the temperatures on the surface of the object and at a first measurement point spaced apart from the surface of the object, or between the temperatures on the surface of the reference body and at a second measurement point spaced apart from the surface of the reference body, as well as a data recorder for recording or calculating the first and second temperature differences.

2. The apparatus according to claim 1, characterized in that the apparatus comprises temperature sensors for measuring the temperatures.

3. The apparatus according to one of the preceding claims, characterized in that the apparatus comprises a thermopile for measuring the first or the second temperature difference.

4. The apparatus according to claim 3, characterized in that a compensating body is thermally connected to the thermopile.

5. A method for ascertaining a transpiration rate of an object, the method comprising the following steps:
determining a first temperature difference between the temperatures on a surface of the object and on a surface of a reference body,
determining a second temperature difference between the temperatures on the surface of the object and at a first measurement point spaced apart from the surface of the object, or between the temperatures on the surface of the reference body and at a second measurement point spaced apart from the surface of the reference body,
ascertaining the transpiration rate of the object, taking into account the first and the second temperature difference.

6. The method according to claim 5, characterized in that the transpiration rate of the object is ascertained taking into account a calibration.

7. The method according to claim 5, characterized in that the calibration is carried out on the basis of a gravimetric measurement of the transpiration or of the transpiration rate.

8. The method according to claim 5, characterized in that the first measurement point is spaced apart from the surface of the object by a maximum distance of 10 cm, or that the second measurement point is spaced apart from the surface of the reference body by a maximum distance of 10 cm.

9. The method according to claim 5, characterized in that the object is a plant leaf or a plant needle.

10. The method according to claim 5, characterized in that the object and the reference body differ from each other
with respect to a dimension,
with respect to an absorption coefficient at a specific wavelength or in a specific wavelength range,
with respect to a thermal capacity and/or
with respect to a coefficient of thermal conductivity
by a maximum of 20% in each case.

11. The method according to claim 10, characterized in that the object and the reference body differ from each other by a maximum of 10%.
